(19) United States
(12) Patent Application Publication
(10) Pub. No.: US 2024/0052104 A1
(43) Pub. Date: Feb. 15, 2024

(54) DICARBONYL HALIDES, POLYMER COMPOSITIONS AND FILMS MADE THEREFROM

(71) Applicant: DUPONT ELECTRONICS, INC., WILMINGTON, DE (US)

(72) Inventors: GREG A. HOSTETLER, WILMINGTON, DE (US); KOSTANTINOS KOURTAKIS, WILMINGTON, DE (US)

(21) Appl. No.: 18/342,108

(22) Filed: Jun. 27, 2023

Related U.S. Application Data
(60) Provisional application No. 63/370,540, filed on Aug. 5, 2022.

(51) Int. Cl. C08G 73/16; C08G 73/14; C08J 5/18; C07C 55/36

(52) U.S. Cl. CPC C08G 73/16; C08G 73/14; C08J 5/18; C07C 55/36; C08J 2379/08

(57) ABSTRACT

In a first aspect, a dicarbonyl halide includes an alicyclic group. In a second aspect, a polymer composition is derived from the dicarbonyl halide, a dianhydride and a diamine, wherein the polymer composition is a poly(amide-imide) or a poly(amide-ester-imide), wherein the poly(amide-ester-imide) is further derived from a polyol. In a third aspect, a polymer film includes the polymer composition. In a fourth aspect, an electronic device includes the polymer film. In a fifth aspect, a metal-clad laminate includes the polymer film.

### FIELD OF DISCLOSURE

The field of this disclosure is dicarbonyl halides, polymer compositions and films made therefrom.

### BACKGROUND OF THE DISCLOSURE

Polymer films, such as polyimide films, are used in a broad range of applications in the electronics industry, taking advantage of the wide variety of mechanical, electrical and optical properties they may provide, as well beneficial thermal and chemical durability needed both during processing of various electronic components and during use of electronic devices. Polymer films can be used in the manufacture of flexible circuits and copper-clad laminates, as well as in display devices, such as for cover windows, touch sensor panels and other device layers. Achieving the desired combination of these properties in a single film, however, can be challenging.

Polymer films having high temperature stability, high tensile modulus and low coefficient of thermal expansion (CTE) are needed for flexible display applications, such as for thin-film transistor (TFT) substrates in organic light-emitting diode (OLED) displays, electronic paper (E-paper) and touch sensor panels (TSPs) for displays.

Polyimide films can potentially replace rigid glass cover sheets and other substrates which are currently used in display applications, such as organic light-emitting diode (OLED) displays. For polyimide films used in display applications, in addition to having high transmittance and low haze, the polyimide film also needs to be neutral in color. Typical specifications require that both a* and b* are no greater than 1 color unit from neutral (0) in CIE L*, a*, b* color space coordinates, i.e., the absolute values of a* and b* should be less than 1. The three coordinates of CIE L*, a*, b* represent: (1) the lightness of the color (L*=0 yields black and L*=100 indicates diffuse white), (2) its position between red/magenta and green (negative a* values indicate green, while positive values indicate magenta) and (3) its position between yellow and blue (negative b* values indicate blue and positive values indicate yellow).

Typical aromatic polyimides with fluorinated monomers, which are nearly colorless, still absorb light in the blue or violet wavelengths (400-450 nm) which gives the films a yellow appearance in transmission. The color of the polyimide films is primarily generated from charge transfer absorptions arising from HOMO-LUMO transitions which can potentially occur both within the polymer chains and between polymer chains. Various approaches have been used to alter HOMO-LUMO transition energies or to frustrate interchain interactions. Although a fluorinated monomer is used to alter the HOMO-LUMO transition energies of the aromatic polyimide polymer, some residual yellow color is apparent in these polyimide films. Depending on the monomer composition in the polyimide, therefore, b* can be higher than 1. Since the CIE L*, a*, b* color measurement of a film is also dependent on its thickness, achieving a neutral appearance is even more difficult for thicker films, such as those greater than 25 μm.

In addition to having good optical properties, polyimide films used in these applications need to maintain good mechanical properties, such as a high elastic modulus. The modulus of a polyimide film can be increased by incorporating more rigid monomers into the polyimide backbone. In the case of rigid aromatic monomers, however, charge transfer absorptions, as described above, lead to higher color for a polyimide incorporating these monomers. Additionally, for rigid non-aromatic monomers, their poor thermal stability at higher temperatures, such as typical imidization temperatures, may lead to decomposition of the monomer, resulting in increased color. These are just two examples of how using more rigid monomers, while improving mechanical properties, may increase the color of polyimides.

Alicyclic and aliphatic monomers, when incorporated into a polyimide structure, can lower color by modifying the electronic structure and charge transfer characteristics of the polymer. These monomers would not, by themselves, participate in any charge transfer transitions. However, a process where the film is formed by casting a polyamic acid solution and curing of the film which is produced results in significant color. The generation of color is more pronounced when curing is performed in air, indicating that a secondary color formation mechanism is occurring.

### DETAILED DESCRIPTION

In a first aspect, a dicarbonyl halide has formula I:

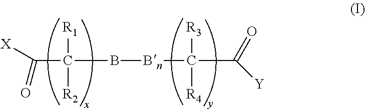

wherein:
   X and Y are the same or different at each occurrence and are independently selected from the group consisting of fluorine, chlorine and bromine;
   R₁-R₄ are the same or different at each occurrence and are independently selected from the group consisting of hydrogen and alkyl, wherein alkyl is C1 to C12 and linear or branched;
   B and B′ are the same or different at each occurrence and are an alicyclic group or an aromatic group with the proviso that at least one of B and B′ is an alicyclic group;
   x and y are the same or different and are an integer from 0 to 12; and

n is an integer from 0 to 4.

In a second aspect, a polymer composition is derived from the dicarbonyl halide, a dianhydride and a diamine, wherein the polymer composition is a poly(amide-imide) or a poly(amide-ester-imide), wherein the poly(amide-ester-imide) is further derived from a polyol.

In a third aspect, a polymer film includes the polymer composition.

In a fourth aspect, an electronic device includes the polymer film.

In a fifth aspect, a metal-clad laminate includes the polymer film.

Depending upon context, “diamine” as used herein is intended to mean: (i) the unreacted form (i.e., a diamine monomer); (ii) a partially reacted form (i.e., the portion or portions of an oligomer or other polymer precursor derived from or otherwise attributable to diamine monomer) or (iii) a fully reacted form (the portion or portions of the polymer derived from or otherwise attributable to diamine monomer). The diamine can be functionalized with one or more moieties, depending upon the particular embodiment selected in the practice of the present invention.

Indeed, the term “diamine” is not intended to be limiting (or interpreted literally) as to the number of amine moieties in the diamine component. For example, (ii) and (iii) above include polymeric materials that may have two, one, or zero amine moieties. Alternatively, the diamine may be functionalized with additional amine moieties (in addition to the amine moieties at the ends of the monomer that react with dianhydride to propagate a polymeric chain). Such additional amine moieties could be used to crosslink the polymer or to provide other functionality to the polymer.

Similarly, the term “dianhydride” as used herein is intended to mean the component that reacts with (is complimentary to) the diamine and in combination is capable of reacting to form an intermediate (which can then be cured into a polymer). Depending upon context, “anhydride” as used herein can mean not only an anhydride moiety per se, but also a precursor to an anhydride moiety, such as: (i) a pair of carboxylic acid groups (which can be converted to anhydride by a de-watering or similar-type reaction); or (ii) an acid halide (e.g., chloride) ester functionality (or any other functionality presently known or developed in the future which is) capable of conversion to anhydride functionality.

Depending upon context, “dianhydride” can mean: (i) the unreacted form (i.e. a dianhydride monomer, whether the anhydride functionality is in a true anhydride form or a precursor anhydride form, as discussed in the prior above paragraph); (ii) a partially reacted form (i.e., the portion or portions of an oligomer or other partially reacted or precursor polymer composition reacted from or otherwise attributable to dianhydride monomer) or (iii) a fully reacted form (the portion or portions of the polymer derived from or otherwise attributable to dianhydride monomer).

The dianhydride can be functionalized with one or more moieties, depending upon the particular embodiment selected in the practice of the present invention. Indeed, the term “dianhydride” is not intended to be limiting (or interpreted literally) as to the number of anhydride moieties in the dianhydride component. For example, (i), (ii) and (iii) (in the paragraph above) include organic substances that may have two, one, or zero anhydride moieties, depending upon whether the anhydride is in a precursor state or a reacted state. Alternatively, the dianhydride component may be functionalized with additional anhydride type moieties (in addition to the anhydride moieties that react with diamine to provide a polymer). Such additional anhydride moieties could be used to crosslink the polymer or to provide other functionality to the polymer.

Any one of a number of polymer manufacturing processes may be used to prepare polymer films. It would be impossible to discuss or describe all possible manufacturing processes useful in the practice of the present invention. It should be appreciated that the monomer systems of the present invention are capable of providing the above-described advantageous properties in a variety of manufacturing processes. The compositions of the present invention can be manufactured as described herein and can be readily manufactured in any one of many (perhaps countless) ways of those of ordinarily skilled in the art, using any conventional or non-conventional manufacturing technology.

Although methods and materials similar or equivalent to those described herein can be used in the practice or testing of the present invention, suitable methods and materials are described herein.

When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In describing certain polymers, it should be understood that sometimes applicants are referring to the polymers by the monomers used to make them or the amounts of the monomers used to make them. While such a description may not include the specific nomenclature used to describe the final polymer or may not contain product-by-process terminology, any such reference to monomers and amounts should be interpreted to mean that the polymer is made from those monomers or that amount of the monomers, and the corresponding polymers and compositions thereof.

The materials, methods, and examples herein are illustrative only and, except as specifically stated, are not intended to be limiting.

As used herein, the terms “comprises,” “comprising,” “includes,” “including,” “has,” “having” or any other variation thereof, are intended to cover a non-exclusive inclusion. For example, a method, process, article, or apparatus that comprises a list of elements is not necessarily limited only those elements but may include other elements not expressly listed or inherent to such method, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

It will be understood that although the terms first, second, third, etc. may be used herein to describe various elements, components, regions, layers and/or sections, these elements, components, regions, layers, and/or sections should not be limited by these terms. These terms are only used to distinguish one element, component, region, layer and/or section from another element, component, region, layer and/or section. Thus, a first element, component, region, layer and/or section could be termed a second element, component, region, layer and/or section without departing from the teachings of the present invention. Similarly, the terms “top” and “bottom” are only relative to each other. It will be appreciated that when an element, component, layer or the like is inverted, what is the “bottom” before being inverted would be the “top” after being inverted, and vice versa. When an element is referred to as being “on” or “disposed on” another element, it means positioning on or below the object portion, but does not essentially mean positioning on the upper side of the object portion based on a gravity direction, and it can be directly on the other element or intervening elements may be present therebetween. In contrast, when an element is referred to as being “directly on” or “disposed directly on” another element, there are no intervening elements present.

Further, it will also be understood that when one element, component, region, layer and/or section is referred to as being “between” two elements, components, regions, layers and/or sections, it can be the only element, component, region, layer and/or section between the two elements, components, regions, layers and/or sections, or one or more intervening elements, components, regions, layers and/or sections may also be present.

### Organic Solvents

Useful organic solvents for the synthesis of the polymers of the present invention are preferably capable of dissolving the polymer precursor materials. Such a solvent should also have a relatively low boiling point, such as below 225° C., so the polymer can be dried at moderate (i.e., more convenient and less costly) temperatures. A boiling point of less than 210, 205, 200, 195, 190, or 180° C. is preferred.

Useful organic solvents include: N-methylpyrrolidone (NMP), dimethylacetamide (DMAc), methyl ethyl ketone (MEK), dimethylformamide (DMF), dimethyl sulfoxide (DMSO), tetramethylurea (TMU), glycol ethyl ether, diethyleneglycol diethyl ether, 1,2-dimethoxyethane (monoglyme), diethylene glycol dimethyl ether (diglyme), 1,2-bis(2-methoxyethoxy)ethane (triglyme), gamma-butyrolactone, and bis(2-methoxyethyl)ether, tetrahydrofuran (THF), ethyl acetate, hydroxyethyl acetate glycol monoacetate, acetone and mixtures thereof. In one embodiment, preferred solvents include N-methylpyrrolidone (NMP) and dimethylacetamide (DMAc).

### Dicarbonyl Halides

In one embodiment, a suitable dicarbonyl halide for forming the polymer can have the formula I:

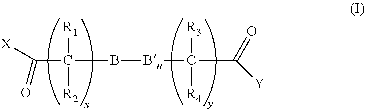

wherein:
   X and Y are the same or different at each occurrence and are independently selected from the group consisting of fluorine, chlorine and bromine;
   R₁-R₄ are the same or different at each occurrence and are independently selected from the group consisting of hydrogen and alkyl, wherein alkyl is C1 to C12 and linear or branched;
   B and B′ are the same or different at each occurrence and are an alicyclic group or an aromatic group with the proviso that at least one of B and B′ is an alicyclic group;
   x and y are the same or different and are an integer from 0 to 12; and
   n is an integer from 0 to 4.

In one embodiment, an alicyclic group of B and B′ can include a phenyl group having two linking bonds, wherein the two linking bonds can be in an ortho, meta or para position relative to one another, for example:

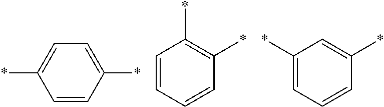

In one embodiment, an alicyclic group of B and B′ can include a saturated carbon ring structure having from three to sixteen carbons (C3 to C16) and two linking bonds, wherein each of the two linking bonds can be attached to any carbon on the ring, for example:

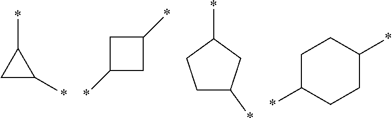

In a specific embodiment, n=0 and B is a saturated C4 to C9 alicyclic group. In a more specific embodiment, n=0 and B is a saturated C6 alicyclic group.

In one embodiment, an alicyclic group of B and B′ can include an unsaturated carbon ring structure having from five to twenty-two carbons (C5 to C22), one or more unsaturated bonds at any position in the ring structure, provided that the ring structure is non-aromatic, and two linking bonds, wherein each of the two linking bonds can be attached to any carbon on the ring, for example:

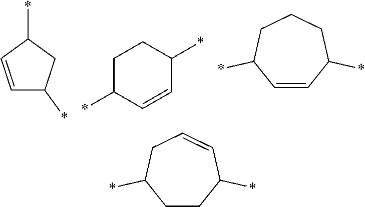

In one embodiment, an alicyclic group of B and B′ can include a bicyclic group, such as a saturated or unsaturated structure having seven or eight carbons (C7 or C8), for example:

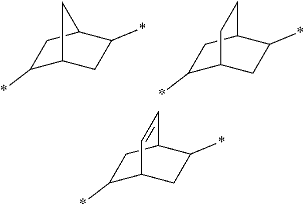

The term “non-aromatic” as used herein to describe an alicyclic carbon ring structure is intended to mean that at least one carbon in the ring has two single bonds to neighboring carbons in the ring, i.e., the ring has at least one quaternary carbon.

In one embodiment, to prepare a dicarbonyl chloride (i.e., X=Y=chlorine), a dicarboxylic acid in dichloromethane (DCM) can be treated with a chlorinating reagent, such as oxalyl or thionyl chloride, followed by a catalytic amount of dimethylformamide (DMF). The mixture can be heated at a sufficient external temperature (e.g., 63° C.) for a sufficient amount of time (e.g., several hours) whereupon the reaction can be concentrated in vacuo to provide a solid. Recrystallization from a suitable solvent (e.g., hexanes) provides a purified product. In another embodiment, a brominating reagent, such as phosphorus tribromide or dibromotriphenylphosphorane, can be used in place of the chlorinating reagent to produce a dicarbonyl bromide (i.e., X=Y=bromine). In still another embodiment, a fluorinating reagent, such as 4-tert-butyl-2,6-dimethylphenylsulfur trifluoride or (diethylamino)sulfur trifluoride, can be used to produce a dicarbonyl fluoride (i.e., X=Y=fluorine).

### Diamines

In one embodiment, a suitable diamine for forming the polymer can include an aliphatic diamine, such as 1,2-diaminoethane, 1,6-diaminohexane (HMD), 1,4-diaminobutane, 1,5-diaminopentane, 1,7-diaminoheptane, 1,8-diaminooctane, 1,9-diaminononane, 1,10-diaminodecane (DMD), 1,11-diaminoundecane, 1,12-diaminododecane (DDD), 1,16-hexadecamethylenediamine, 1,3-bis(3-aminopropyl)-tetramethyldisiloxane, trans-1,4-cyclohexanediamine (CHDA), isophoronediamine (IPDA), bicyclo[2.2.2]octane-1,4-diamine and combinations thereof. Other aliphatic diamines suitable for practicing the invention include those having six to twelve carbon atoms or a combination of longer chain and shorter chain diamines so long as both developability and flexibility of the polymer are maintained. Long chain aliphatic diamines may increase flexibility.

In one embodiment, a suitable diamine for forming the polymer can include an alicyclic diamine (can be fully or partially saturated), such as a cyclobutane diamine (e.g., cis- and trans-1,3-diaminocyclobutane, 6-amino-3-azaspiro[3.3]heptane, and 3,6-diaminospiro[3.3]heptane), bicyclo[2.2.1]heptane-1,4-diamine, isophoronediamine, and bicyclo[2.2.2]octane-1,4-diamine. Other alicyclic diamines can include cis-1,4-cyclohexanediamine, trans-1,4-cyclohexanediamine, 1,4-bis(aminomethyl)cyclohexane, 4,4′-methylenebis(cyclohexylamine), 4,4′-methylenebis(2-methylcyclohexylamine) and bis(aminomethyl)norbornane.

In one embodiment, a suitable diamine for forming the polymer can include a fluorinated aromatic diamine, such as 2,2′-bis(trifluoromethyl)benzidine (TFMB), trifluoromethyl-2,4-diaminobenzene, trifluoromethyl-3,5-diaminobenzene, 2,2′-bis(4-aminophenyl)hexafluoropropane, 4,4′-diamino-2,2′-trifluoromethyldiphenyloxide, 3,3′-diamino-5,5′-trifluoromethyldiphenyloxide, 9,9′-bis(4-aminophenyl)fluorene, 4,4′-trifluoromethyl-2,2′-diaminobiphenyl, 4,4′-oxy-bis[(2-trifluoromethyl)benzeneamine] (1,2,4-OBABTF), 4,4′-oxy-bis[(3-trifluoromethyl)benzeneamine], 4,4′-thio-bis[(2-trifluoromethyl)benzeneamine], 4,4′-thiobis[(3-trifluoromethyl)benzeneamine], 4,4′-sulfoxyl-bis[(2-trifluoromethyl)benzeneamine], 4,4′-sulfoxyl-bis[(3-trifluoromethyl)benzeneamine], 4,4′-keto-bis[(2-trifluoromethyl)benzeneamine], 1,1-bis[4′-(4″-amino-2″-trifluoromethylphenoxy)phenyl]cyclopentane, 1,1-bis[4′-(4″-amino-2″-trifluoromethylphenoxy)phenyl]cyclohexane, 2-trifluoromethyl-4,4′-diaminodiphenylether, 1,4-(2′-trifluoromethyl-4′,4″-diaminodiphenoxy)benzene, 1,4-bis(4′-aminophenoxy)-2-[(3′,5′-ditrifluoromethyl)phenyl]benzene, 1,4-bis[2′-cyano-3′-(″4-aminophenoxy)phenoxy]-2-[(3′,5′-ditrifluoromethyl)phenyl]benzene (6FC-diamine), 3,5-diamino-4-methyl-2′,3′,5′,6′-tetrafluoro-4′-trifluoromethyldiphenyloxide, 2,2-bis[4′(4″-aminophenoxy)phenyl]phthalein-3′,5′-bis(trifluoromethyl)anilide (6FADAP) and 3,3′,5,5′-tetrafluoro-4,4′-diamino-diphenylmethane (TFDAM).

Other useful diamines for forming the polymer can include p-phenylenediamine (PPD), m-phenylenediamine (MPD), 2,5-dimethyl-1,4-diaminobenzene, 2,5-dimethyl-1,4-phenylenediamine (DPX), 2,2-bis(4-aminophenyl)propane, 1,4-naphthalenediamine, 1,5-naphthalenediamine, 4,4′-diaminobiphenyl, 4,4″-diaminoterphenyl, 4,4′-diaminobenzanilide, 4,4′-diaminophenylbenzoate, 4,4′-diaminobenzophenone, 4,4′-diaminodiphenylmethane (MDA), 4,4′-diaminodiphenylsulfide, 4,4′-diaminodiphenylsulfone, 3,3′-diaminodiphenylsulfone, bis[4-(4-aminophenoxy)phenyl]sulfone (BAPS), 4,4′-bis(aminophenoxy)biphenyl (BAPB), 4,4′-diaminodiphenylether (ODA), 3,4′-diaminodiphenylether, 4,4′-diaminobenzophenone, 4,4′-isopropylidenedianiline, 2,2′-bis(3-aminophenyl)propane, N,N-bis(4-aminophenyl)-n-butylamine, N,N-bis(4-aminophenyl)methylamine, 1,5-diaminonaphthalene, 3,3′-dimethyl-4,4′-diaminobiphenyl, m-aminobenzoyl-p-aminoanilide, 4-aminophenyl-3-aminobenzoate, N,N-bis(4-aminophenyl)aniline, 2,4-diaminotoluene, 2,5-diaminotoluene, 2,6-diaminotoluene, 2,4-diamine-5-chlorotoluene, 2,4-diamine-6-chlorotoluene, 2,4-bis-(beta-amino-t-butyl)toluene, bis-(p-beta-amino-t-butylphenyl)ether, p-bis-2-(2-methyl-4-aminopentyl)benzene, m-xylylenediamine and p-xylylenediamine.

Other useful diamines for forming the polymer can include 1,2-bis(4-aminophenoxy)benzene, 1,3-bis(4-aminophenoxy)benzene (RODA), 1,2-bis(3-aminophenoxy)benzene, 1,3-bis(3-aminophenoxy)benzene, 1-(4-aminophenoxy)-3-(3-aminophenoxy)benzene, 1,4-bis(4-aminophenoxy)benzene, 1,4-bis(3-aminophenoxy)benzene, 1-(4-aminophenoxy)-4-(3-aminophenoxy)benzene, 2,2-bis(4-[4-aminophenoxy]phenyl)propane (BAPP), 2,2′-bis(4-phenoxyaniline)isopropylidene, 2,4,6-trimethyl-1,3-diaminobenzene and 2,4,6-trimethyl-1,3-diaminobenzene.

### Dianhydrides

In one embodiment, any number of suitable dianhydrides can be used in forming the polymer. The dianhydrides can be used in their tetra-acid form (or as mono, di, tri, or tetra esters of the tetra acid), or as their diester acid halides (chlorides). However, in some embodiments, the dianhydride form can be preferred, because it is generally more reactive than the acid or the ester.

Examples of suitable dianhydrides include 3,3′,4,4′-biphenyltetracarboxylic dianhydride (BPDA), 1,2,5,6-naphthalenetetracarboxylic dianhydride, 1,4,5,8-naphthalenetetracarboxylic dianhydride, 2,3,6,7-naphthalenetetracarboxylic dianhydride, 2-(3′,4′-dicarboxyphenyl)-5,6-dicarboxybenzimidazole dianhydride, 2-(3′,4′-dicarboxyphenyl)-5,6-dicarboxybenzoxazole dianhydride, 2-(3′,4′-dicarboxyphenyl)-5,6-dicarboxybenzothiazole dianhydride, 2,2′,3,3′-benzophenonetetracarboxylic dianhydride, 2,3,3′,4′-benzophenonetetracarboxylic dianhydride, 3,3′,4,4′-benzophenonetetracarboxylic dianhydride (BTDA), 2,2′,3,3′-biphenyltetracarboxylic dianhydride, 2,3,3′,4′-biphenyltetracarboxylic dianhydride, bicyclo[2.2.2]oct-7-ene-2,3,5,6-tetracarboxylic-2,3,5,6-dianhydride, 4,4′-thiodiphthalic anhydride, bis(3,4-dicarboxyphenyl)sulfone dianhydride, bis(3,4-dicarboxyphenyl)sulfoxide dianhydride (DSDA), bis(3,4-dicarboxyphenyloxadiazole-1,3,4)-p-phenylene dianhydride, bis(3,4-dicarboxyphenyl)-2,5-oxadiazole-1,3,4-dianhydride, bis-2,5-(3′,4′-dicarboxydiphenylether)-1,3,4-oxadiazole dianhydride, 4,4′-oxydiphthalic anhydride (ODPA), bis(3,4-dicarboxyphenyl)thioether dianhydride, bisphenol A dianhydride (BPADA), bisphenol S dianhydride, bis-1,3-isobenzofurandione, 1,4-bis(4,4′-oxyphthalic anhydride)benzene, bis(3,4-dicarboxyphenyl)methane dianhydride, cyclopentadienyltetracarboxylic dianhydride, ethylenetetracarboxylic dianhydride, perylene-3,4,9,10-tetracarboxylic dianhydride, pyromellitic dianhydride (PMDA), tetrahydrofurantetracarboxylic dianhydride, 1,3-bis(4,4′-oxydiphthalic anhydride)benzene, 2,2-bis(3,4-dicarboxyphenyl)propane dianhydride, 2,6-dichloronaphthalene-1,4,5,8-tetracarboxylic dianhydride, 2,7-dichloronaphthalene-1,4,5,8-tetracarboxylic dianhydride, 2,3,6,7-tetrachloronaphthalene-1,4,5,8-tetracarboxylic dianhydride, phenanthrene-1,8,9,10-tetracarboxylic dianhydride, pyrazine-2,3,5,6-tetracarboxylic dianhydride, benzene-1,2,3,4-tetracarboxylic dianhydride and thiophene-2,3,4,5-tetracarboxylic dianhydride.

In one embodiment, a suitable dianhydride can include an alicyclic dianhydride, such as cyclobutane-1,2,3,4-tetracarboxylic dianhydride (CBDA), 1,2,4,5-cyclohexanetetracarboxylic dianhydride, 1,2,3,4-cyclohexanetetracarboxylic dianhydride, 1,2,3,4-tetramethyl-1,2,3,4-cyclobutanetetracarboxylic dianhydride, 1,2,3,4-cyclopentanetetracarboxylic dianhydride (CPDA), hexahydro-4,8-ethano-1H,3H-benzo[1,2-c:4,5-c′]difuran-1,3,5,7-tetrone (BODA), 3-(carboxymethyl)-1,2,4-cyclopentanetricarboxylic 1,4:2,3-dianhydride (TCA) and meso-butane-1,2,3,4-tetracarboxylic dianhydride. In one embodiment, an alicyclic dianhydride can be present in an amount of about 70 mole percent or less, based on the total dianhydride content of the polymer.

In one embodiment, a suitable dianhydride for forming the polymer can include a fluorinated dianhydride, such as 4,4′-(hexafluoroisopropylidene)diphthalic anhydride (6FDA) and 9,9-bis(trifluoromethyl)-2,3,6,7-xanthenetetracarboxylic dianhydride.

In one embodiment, wherein the polymer further includes additional aromatic amide components, a suitable additional dicarbonyl chloride for forming the polymer can include terephthaloyl chloride (TPCl), isophthaloyl chloride (IPCl), biphenyl dicarbonyl chloride (BPCl), naphthalene dicarbonyl chloride, terphenyl dicarbonyl chloride, 2-fluoro-terephthaloyl chloride and trimellitic anhydride.

In one embodiment, poly(amide-ester-imides) can be produced from polyols which can react with carboxylic acid or the ester acid halides to generate ester linkages.

The dihydric alcohol component may be almost any alcoholic diol containing two esterifiable hydroxyl groups. Mixtures of suitable diols may also be included. Suitable diols for use herein include for example, ethylene glycol, propylene glycol, 1,4-butanediol, 1,5-pentanediol, neopentyl glycol, etc.

The polyhydric alcohol component may be almost any polyhydric alcohol containing at least 3 esterifiable hydroxyl groups in order to provide the above-described synthesis process advantages of this invention. Mixtures of such polyhydric alcohols may suitably be employed. Suitable polyhydric alcohols include, for example, tris(2-hydroxyethyl)isocyanurate, glycerin, 1,1,1-trimethylolethane, 1,1,1-trimethylolpropane, and their mixtures.

In some cases, useful diamine and dianhydride monomers contain ester groups. Examples of these monomers include diamines, such as 4-aminophenyl-4-aminobenzoate and 4-amino-3-methylphenyl-4-aminobenzoate, and dianhydrides, such as p-phenylene bis(trimellitate) dianhydride.

In some cases, useful diamine and dianhydride monomers contain amide groups. Examples of these monomers include diamines, such as 4,4′-diaminobenzamide (DABAN), and dianhydrides, such as N,N′-(2,2′-bis(trifluoromethyl)-[1,1′-biphenyl]-4,4′-diyl)bis(1,3-dioxo-1,3-dihydroisobenzofuran-5-carboxamide) and N,N′-(9H-fluoren-9-ylidenedi-4,1-phenylene)bis[1,3-dihydro-1,3-dioxo-5-isobenzofurancarboxamide].

Higher order copolymers having an imide group can include any of the monomers described above.

### Polymer Films

Polymer films made from poly(amide-imide) and poly(amide-ester-imide) compositions of the present invention can have improved properties compared to polyimide films and can be useful in a wide range of applications. For example, improvements in mechanical properties can result from interchain interactions of the amide-containing polymer backbones. One such interaction is hydrogen bonding of the amide protons on one chain to moieties on neighboring chains. These interactions typically lead to improved modulus and in some cases, can lead to lower coefficient of thermal expansion (CTE), improved tear resistance and resistance to micro-cracking. The modulus and CTE improvements are especially useful, for example, in flexible printed circuits, solar cell substrates, and thin film transistor substrates. The combination of using the alicyclic groups, which can disrupt charge transfer which would otherwise lead to color, and low temperature processing can further improve the optical properties of these compositions, which can be very useful for colorless TFT substrates, cover windows, and touch sensor panel substrate applications in foldable displays.

In one embodiment, a polymer film containing a poly(amide-imide) composition can be produced by combining a dicarbonyl halide, a diamine and a dianhydride (monomer or other polymer precursor form) together with a solvent to form a poly(amide-amic) acid solution. The molecular weight of the poly(amide-amic) acid formed therefrom can be adjusted by adjusting the molar ratio of the dicarbonyl halide and dianhydride relative to the diamine. In one embodiment, a polymer film containing a poly(amide-ester-imide) composition can be produced by combining a dicarbonyl halide, a polyol, a diamine and a dianhydride (monomer or other polymer precursor form) together with a solvent to form a poly(amide-ester-amic) acid solution.

In one embodiment, a poly(amide-amic) acid casting solution is derived from a poly(amide-amic) acid solution. The poly(amide-amic) acid casting solution, and/or the poly(amide-amic) acid solution, can optionally be combined with conversion chemicals like: (i) one or more dehydrating agents, such as, aliphatic acid anhydrides (acetic anhydride, etc.) and/or aromatic acid anhydrides; and (ii) one or more catalysts, such as, aliphatic tertiary amines (triethylamine, etc.), aromatic tertiary amines (N,N-dimethylaniline, etc.) and heterocyclic tertiary amines (pyridine, picoline, isoquinoline, etc.). The anhydride dehydrating material it is often used in molar excess compared to the amount of amide acid groups in the poly(amide-amic) acid. The amount of acetic anhydride used is typically about 2.0-4.0 moles per equivalent (repeat unit) of poly(amide-amic) acid. Generally, a comparable amount of tertiary amine catalyst is used.

In one embodiment, a conversion chemical can be an imidization catalyst (sometimes called an “imidization accelerator”) that can help lower the imidization temperature and shorten the imidization time. Typical imidization catalysts can range from bases such as imidazole, 1-methylimidazole, 2-methylimidazole, 1,2-dimethylimidazole, 2-phenylimidazole, benzimidazole, isoquinoline, substituted pyridines such as methyl pyridines, lutidine, and trialkylamines and hydroxy acids such as isomers of hydroxybenzoic acid. The ratio of these catalysts and their concentration in the poly(amide-amic) acid layer will influence imidization kinetics and the film properties.

In one embodiment, the poly(amide-amic) acid solution, and/or the poly(amide-amic) acid casting solution, is dissolved in an organic solvent at a concentration from about 5.0 or 10% to about 15, 20, 25, 30, 35 or 40% by weight.

The solvated mixture (the poly(amide-amic) acid casting solution) can then be cast or applied onto a support, such as an endless belt or rotating drum, to give a film. Alternatively, it can be cast on a polymeric carrier such as PET, other forms of Kapton® Next, the solvent-containing film can be converted into a self-supporting film by heating at an appropriate temperature (thermal curing). The film can then be separated from the support, oriented such as by tentering, with continued heating (drying and curing) to provide a polymer film.

Useful methods for producing polymer films containing a poly(amide-imide) or a poly(amide-ester-imide) can be found in U.S. Pat. Nos. 5,166,308 and 5,298,331, which are incorporate by reference into this specification for all teachings therein. Numerous variations are also possible and can be used in the current invention, such as,
(a) A method wherein the diamine, dianhydride and dicarbonyl halide components are preliminarily mixed together and then the mixture is added in portions to a solvent while stirring.
(b) A method wherein a solvent is added to a stirring mixture of diamine and dianhydride and dicarbonyl halide components. (contrary to (a) above)
(c) A method wherein diamines are exclusively dissolved in a solvent and then dianhydrides and dicarbonyl halides are added thereto at such a ratio as allowing to control the reaction rate.
(d) A method wherein the dianhydride and dicarbonyl halide components are exclusively dissolved or dispersed in a solvent and then amine components are added thereto at such a ratio to allow control of the reaction rate.
(e) A method wherein the diamine, dianhydride and dicarbonyl halide components are separately dissolved or dispersed in solvents and then these solutions are mixed in a reactor.
(f) A method wherein the poly(amide-amic) acid with excessive amine component and another poly(amide-amic) acid with excessive dianhydride or dicarbonyl halide component are preliminarily formed and then reacted with each other in a reactor, particularly in such a way as to create a non-random or block copolymer.
(g) A method wherein a specific portion of the amine, dianhydride or dicarbonyl halide components are first reacted and then the residual diamine components are reacted, or vice versa.
(h) A method wherein the conversion chemicals (catalysts) are mixed with the poly(amide-amic) acid to form a poly(amide-amic) acid casting solution and then cast to form a gel film.
(i) A method wherein the components are added in part or in whole in any order to either part or whole of the solvent, also where part or all of any component can be added as a solution in part or all of the solvent.
(j) A method of first reacting one of the dianhydride and dicarbonyl halide components with one of the diamine components giving a first poly(amide-amic) acid. Then reacting another dianhydride and dicarbonyl halide component with another amine component to give a second poly(amide-amic) acid. Then combining the poly(amide-amic) acids in any one of a number of ways prior to film formation.

In one embodiment, the poly(amide-amic) acid solution can be heated, optionally in the presence of an imidization catalyst, to partially or fully imidize the poly(amide-amic) acid, converting it to a polymer having an imide group. Temperature, time, and the concentration and choice of imidization catalyst can impact the degree of imidization of the poly(amide-amic) acid solution. Preferably, the solution should be substantially imidized. In one embodiment, for a substantially polymerized solution, greater than 85%, greater than 90%, or greater than 95% of the amic acid groups are converted to the polymer having an imide group, as determined by infrared spectroscopy.

In one embodiment, the solvated mixture (the substantially imidized solution) can be cast to form a polymer film. In another embodiment, the solvated mixture (the first substantially imidized solution) can be precipitated with an antisolvent, such as water or alcohols (e.g., methanol, ethanol, isopropyl alcohol), and the solid polymer resin can be isolated. For instance, isolation can be achieved through filtration, decantation, centrifugation and decantation of the supernatant liquid, distillation or solvent removal in the vapor phase, or by other known methods for isolating a solid precipitate from a slurry. In one embodiment, the precipitate can be washed to remove the catalyst. After washing, the precipitate may be substantially dried, but need not be completely dry. The polymer precipitate can be re-dissolved in a second solvent, such as methyl isobutyl ketone (MIBK), methyl ethyl ketone (MEK), ethyl acetate, methyl acetate, ethyl formate, methyl formate, tetrahydrofuran, acetone, DMAc, NMP and mixtures thereof, to form a second substantially imidized solution (a casting solution), which can be cast to form a polymer film.

In one embodiment, a substantially polymerized solution is formed using monomers (diamines, dianhydrides or dicarbonyl halides) with structural characteristics important for solubility, including flexible linkages, such as, but not limited to, aliphatic spacers, ethers, thioethers, substituted amines, amides, esters, and ketones, weak intermolecular interactions, bulky substitutions, non-coplanarity, non-linearity and asymmetry.

In one embodiment, a solvated mixture (a substantially imidized solution) can be mixed with a crosslinking precursor and a colorant, such as a pigment or a dye, and then cast to form a polymer film. In one embodiment, the colorant may be a low conductivity carbon black. In another embodiment, a solvated mixture (a first substantially imidized solution) can be precipitated with an antisolvent, such as water or alcohols (e.g., methanol, ethanol, isopropyl alcohol). In one embodiment, the precipitate can be washed to remove the catalyst. After washing, the precipitate may be substantially dried, but need not be completely dry. The polymer precipitate can be re-dissolved in a second solvent, such as methyl isobutyl ketone (MIBK), methyl ethyl ketone (MEK), tetrahydrofuran (THF), cyclopentanone, ethyl acetate, acetone, DMAc, NMP and mixtures thereof, to form a second substantially imidized solution (a casting solution). To the second substantially imidized solution, a crosslinking precursor and a colorant can be added, which can then be cast to form a polymer film. In one embodiment, a polymer film contains a crosslinked polymer in a range of from about 80 to about 99 wt %. In some embodiments, the polymer film contains crosslinked polymer in a range of about between and including any two of the following: 80, 85, 90, 95 and 99 wt %. In yet another embodiment, the polymer film contains about 91 to about 98 wt % crosslinked polymer.

In one embodiment, a substantially imidized polymer solution can be cast or applied onto a support, such as an endless belt or rotating drum, to form a film. Alternatively, it can be cast on a polymeric carrier such as PET, other forms of Kapton® Next, the solvent-containing film can be converted into a film by heating to partially or fully remove the solvent. In some aspects of the invention, the film is separated from the carrier before drying to completion. Final drying steps can be performed with dimensional support or stabilization of the film. In other aspects, the film is heated directly on the carrier.

The casting solution can further comprise any one of a number of additives, such as processing aids (e.g., oligomers), antioxidants, light stabilizers, flame retardant additives, anti-static agents, heat stabilizers, ultraviolet absorbing agents, inorganic fillers or various reinforcing agents. Inorganic fillers can include thermally conductive fillers, metal oxides, inorganic nitrides and metal carbides, and electrically conductive fillers like metals. Common inorganic fillers are alumina, silica, diamond, clay, talc, sepiolite, boron nitride, aluminum nitride, titanium dioxide, dicalcium phosphate, and fumed metal oxides. Low color organic fillers, such as polydialkylfluorenes, can also be used. Common organic fillers include polyaniline, polythiophene, polypyrrole, polyphenylenevinylene, polydialkylfluorenes, carbon black, graphite, multiwalled and single walled carbon nanotubes and carbon nanofibers. In one embodiment, nanoparticle fillers and nanoparticle colloids can be used.

In one embodiment, an electrically conductive filler is carbon black. In one embodiment, the electrically conductive filler is selected from the group consisting of acetylene blacks, super abrasion furnace blacks, conductive furnace blacks, conductive channel type blacks, carbon nanotubes, carbon fibers, fine thermal blacks and mixtures thereof. As described above for low conductivity carbon black, oxygen complexes on the surface of the carbon particles act as an electrically insulating layer. Thus, low volatility content is generally desired for high conductivity. However, it is also necessary to consider the difficulty of dispersing the carbon black. Surface oxidation enhances deagglomeration and dispersion of carbon black. In some embodiments, when the electrically conductive filler is carbon black, the carbon black has a volatile content less than or equal to 1%.

Fillers can have a size of less than 550 nm in at least one dimension. In other embodiments, the filler can have a size of less than 500, less than 450, less than 400, less than 350, less than 300, less than 250, or less than 200 nm (since fillers can have a variety of shapes in any dimension and since filler shape can vary along any dimension, the “at least one dimension” is intended to be a numerical average along that dimension). The average aspect ratio of the filler can be 1 or greater. In some embodiments, the sub-micron filler is selected from a group consisting of needle-like fillers (acicular), fibrous fillers, platelet fillers, polymer fibers, and mixtures thereof. In one embodiment, the sub-micron filler is substantially non-aggregated. The sub-micron filler can be hollow, porous, or solid. In one embodiment, the sub-micron fillers of the present disclosure exhibit an aspect ratio of at least 1, at least 2, at least 4, at least 6, at least 8, at least 10, at least 12, or at least 15 to 1.

In some embodiments, sub-micron fillers are 100 nm in size or less. In some embodiments, the fillers are spherical or oblong in shape and are nanoparticles. In one embodiment, sub-micron fillers can include inorganic oxides, such as oxides of silicon, aluminum and titanium, hollow (porous) silicon oxide, antimony oxide, zirconium oxide, indium tin oxide, antimony tin oxide, mixed titanium/tin/zirconium oxides, and binary, ternary, quaternary and higher order composite oxides of one or more cations selected from silicon, titanium, aluminum, antimony, zirconium, indium, tin, zinc, niobium and tantalum. In one embodiment, nanoparticle composites (e.g. single or multiple core/shell structures) can be used, in which one oxide encapsulates another oxide in one particle.

In one embodiment, sub-micron fillers can include other ceramic compounds, such as boron nitride, aluminum nitride, ternary or higher order compounds containing boron, aluminum and nitrogen, gallium nitride, silicon nitride, aluminum nitride, zinc selenide, zinc sulfide, zinc telluride, silicon carbide, and their combinations, or higher order compounds containing multiple cations and multiple anions.

In one embodiment, solid silicon oxide nanoparticles can be produced from sols of silicon oxides (e.g., colloidal dispersions of solid silicon oxide nanoparticles in liquid media), especially sols of amorphous, semi-crystalline, and/or crystalline silica. Such sols can be prepared by a variety of techniques and in a variety of forms, which include hydrosols (i.e., where water serves as the liquid medium), organosols (i.e., where organic liquids serve as the liquid medium), and mixed sols (i.e., where the liquid medium comprises both water and an organic liquid). See, e.g., descriptions of the techniques and forms disclosed in U.S. Pat. Nos. 2,801,185, 4,522,958 and 5,648,407. In one embodiment, the nanoparticle is suspended in a polar, aprotic solvent, such as, DMAc or other solvent compatible with poly(amide-amic) acid. In another embodiment, solid nanosilica particles can be commercially obtained as colloidal dispersions or sols dispersed in polar aprotic solvents, such as for example DMAC-ST (Nissan Chemical America Corporation, Houston TX), a solid silica colloid in dimethylacetamide containing less than 0.5 percent water, with 20-21 wt % SiO₂, with a median nanosilica particle diameter, d₅₀, of about 16 nm.

In one embodiment, sub-micron fillers can be porous and can have pores of any shape. One example is where the pore comprises a void of lower density and low refractive index (e.g., a void-containing air) formed within a shell of an oxide such as silicon oxide, i.e., a hollow silicon oxide nanoparticle. The thickness of the sub-micron fillers shell affects the strength of the sub-micron fillers. As the hollow silicon oxide particle is rendered to have reduced refractive index and increased porosity, the thickness of the shell decreases resulting in a decrease in the strength (i.e., fracture resistance) of the sub-micron fillers. Methods for producing such hollow silicon oxide nanoparticles are known, for example, as described in Japanese Patent Nos. 4406921B2 and 4031624B2. Hollow silicon oxide nanoparticles can be obtained from JGC Catalysts and Chemicals, LTD, Japan.

In one embodiment, sub-micron fillers can be coated with a coupling agent. For example, a nanoparticle can be coated with an aminosilane, phenylsilane, acrylic or methacrylic coupling agents derived from the corresponding alkoxysilanes. Trimethylsilyl surface capping agents can be introduced to the nanoparticle surface by reaction of the sub-micron fillers with hexamethyldisilazane. In one embodiment, sub-micron fillers can be coated with a dispersant. In one embodiment, sub-micron fillers can be coated with a combination of a coupling agent and a dispersant. Alternatively, the coupling agent, dispersant or a combination thereof can be incorporated directly into the polymer film and not necessarily coated onto the sub-micron fillers.

In some embodiments a coextrusion process can used to form a multilayer polymer film with an inner core layer sandwiched between two outer layers. In this process, a finished poly(amide-amic) acid solution is filtered and pumped to a slot die, where the flow is divided in such a manner as to form the first outer layer and the second outer layer of a three-layer coextruded film. In some embodiments, a second stream of polymer is filtered, then pumped to a casting die, in such a manner as to form the middle core layer of a three-layer coextruded film. The flow rates of the solutions can be adjusted to achieve the desired layer thickness. In one embodiment, the polymers in any of the three layers can be the same or different.

In some embodiments, the multilayer film is prepared by simultaneously extruding the first outer layer, the core layer and the second outer layer. In some embodiments, the layers are extruded through a single or multi-cavity extrusion die. In another embodiment, the multilayer film is produced using a single-cavity die. If a single-cavity die is used, the laminar flow of the streams should be of high enough viscosity to prevent comingling of the streams and to provide even layering. In some embodiments, the multilayer film is prepared by casting from the slot die onto a moving stainless-steel belt. In one embodiment, the belt is then passed through a convective oven, to evaporate solvent and partially imidize the polymer, to produce a “green” film. The green film can be stripped off the casting belt and wound up. The green film can then be passed through a tenter oven to produce a fully cured polymer film. In some embodiments, during tentering, shrinkage can be minimized by constraining the film along the edges (i.e., using clips or pins).

The thickness of the polymer film may be adjusted, depending on the intended purpose of the film or final application specifications. In one embodiment, the polymer film has a total thickness in a range of from about 10 to about 80 μm, or from about 10 to about 25 μm, or from about 15 to about 25 μm.

In one embodiment, the polymer film has a tensile modulus of at least 4.0 GPa, or at least 4.5 GPa, or at least 5.0 GPa, or at least 5.5 GPa, or at least 6.0 GPa, or at least 6.5 GPa.

In one embodiment, the polymer film has a coefficient of thermal expansion (CTE) of 50 ppm/° C. or less, or 45 ppm/° C. or less, or 40 ppm/° C. or less or 35 ppm/° C. or less, or 30 ppm/° C. or less over a temperature range of 50 to 250° C.

In one embodiment, the polymer film has a b* of less than about 1.25, or less than about 1.0 or less than about 0.8 for a film thickness of about 25 μm, when measured with a dual-beam spectrophotometer, using D65 illumination and 10-degree observer, in total transmission mode over a wavelength range of 360 to 780 nm. In one embodiment, the polymer film has a yellowness index (YI) of less than about 2.25, or less than about 2.0 or less than about 1.75 for a film thickness of about 25 μm, when measured using the procedure described by ASTM E313.

In one embodiment, a curable resin coating composition can be applied to a polymer film layer. In some embodiments, a curable resin coating composition can be applied to an article including a polymer film layer and an inorganic substrate, wherein the curable resin coating composition is applied to a surface of the polymer film layer on a side opposite the inorganic substrate. In one embodiment, a curable resin coating composition comprises at least one curable oligomer and at least one organic coating solvent. Suitable curable oligomers are any which form a hard coat layer upon curing. As used herein, the term “hard coat” refers to a material, coating, or layer on a substrate that forms a film upon curing having a higher pencil hardness than the substrate. Such hard coat layers protect the underlying substrate from mechanical abrasion and wear, and optionally enhances the self-cleaning properties of the surface.

Suitable curable oligomers useful in a curable resin coating composition include, but are not limited to, (meth)acrylate oligomers, urethane oligomers, (meth)acrylate-urethane oligomers, siloxane oligomers, and combinations thereof. Liquid curable oligomers are preferred. Suitable (meth)acrylate oligomers include, without limitation, oligomers comprising as polymerized units one or more (meth)acrylate monomers chosen from an aliphatic monofunctional (meth)acrylate monomers and aliphatic multifunctional (meth)acrylate monomers. It is preferred that the present curable oligomer is chosen from (meth)acrylate oligomers, (meth)acrylate-urethane oligomers, siloxane oligomers, and combinations thereof, more preferably from (meth)acrylate-urethane oligomers and a siloxane oligomer.

In some embodiments, the curable resin coating compositions may comprise siloxane oligomers. Suitable siloxane oligomers are those disclosed in U.S. Patent Application Publication Nos. 2015/0159044 and 2017/0369654, and in U.S. Pat. Nos. 7,790,347 and 6,391,999.

### Metal-Clad Laminates

In one embodiment, a conductive layer of the present invention can be created by:
i. metal sputtering (optionally, then electroplating);
ii. foil lamination; and/or
iii. any conventional or non-conventional method for applying a thin metallic layer to a substrate.

Metal-clad laminates can be formed as single-sided laminates or double-sided laminates by any number of well-known processes. In one embodiment, a lamination process may be used to form a metal-clad laminate with a polymer film or multilayer polymer film. In one embodiment, a first outer layer including a first thermoplastic polymer is placed between a first conductive layer and a core layer, and a second outer layer including a second thermoplastic polymer is placed on the opposite side of the core layer. In one embodiment, a second conductive layer is placed in contact with the second outer layer on a side opposite the core layer. One advantage of this type of construction is that the lamination temperature of the multilayer film is lowered to the lamination temperature necessary for the thermoplastic polymer of the outer layer to bond to a conductive layer(s). In one embodiment, the conductive layer(s) is a metal layer(s).

For example, prior to the step of applying a polymer film onto a metal foil, the polymer film can be subjected to a pre-treatment step. Pre-treatment steps can include, heat treatment, corona treatment, plasma treatment under atmospheric pressure, plasma treatment under reduced pressure, treatment with coupling agents like silanes and titanates, sandblasting, alkali-treatment, acid-treatments, and coating polyamic acids. To improve the adhesion strength, it is generally also possible to add various metal compounds as disclosed in U.S. Pat. Nos. 4,742,099; 5,227,244; 5,218,034; and 5,543,222, incorporated herein by reference.

In addition, (for purposes of improving adhesion) the conductive metal surface may be treated with various organic and inorganic treatments. These treatments include using silanes, imidazoles, triazoles, oxide and reduced oxide treatments, tin oxide treatment, and surface cleaning/roughening (called micro-etching) via acid or alkaline reagents.

In a further embodiment, the poly(amide-amic) acid precursor (to a polymer film of the present invention) may be coated on a fully cured polymer base film or directly on a metal substrate and subsequently imidized by heat treatment. The polymer base film may be prepared by either a chemical or thermal conversion process and may be surface treated, e.g., by chemical etching, corona treatment, laser etching etc., to improve adhesion.

As used herein, the term “conductive layers” and “conductive foils” mean metal layers or metal foils (thin compositions having at least 50% of the electrical conductivity of a high-grade copper). Conductive foils are typically metal foils. Metal foils do not have to be used as elements in pure form; they may also be used as metal foil alloys, such as copper alloys containing nickel, chromium, iron, and other metals. The conductive layers may also be alloys of metals and are typically applied to the polymers of the present invention via a sputtering step followed by an electro-plating step. In these types of processes, a metal seed coat layer is first sputtered onto a polymer film. Finally, a thicker coating of metal is applied to the seed coat via electro-plating or electro-deposition. Such sputtered metal layers may also be hot pressed above the glass transition temperature of the polymer for enhanced peel strength.

Particularly suitable metallic substrates are foils of rolled, annealed copper or rolled, annealed copper alloy. In many cases, it has proved to be advantageous to pre-treat the metallic substrate before coating. This pre-treatment may include, but is not limited to, electro-deposition or immersion-deposition on the metal of a thin layer of copper, zinc, chrome, tin, nickel, cobalt, other metals, and alloys of these metals. The pre-treatment may consist of a chemical treatment or a mechanical roughening treatment. It has been found that this pre-treatment enables the adhesion of the polymer layer and, hence, the peel strength to be further increased. Apart from roughening the surface, the chemical pre-treatment may also lead to the formation of metal oxide groups, enabling the adhesion of the metal to a polymer layer to be further increased. This pre-treatment may be applied to both sides of the metal, enabling enhanced adhesion to substrates on both sides.

In one embodiment, a metal-clad laminate can include the polymer film that is a single-layer film or a multilayer film and a first metal layer adhered to an outer surface of the first outer layer of the multilayer film. In one embodiment, a metal-clad laminate can include a second metal layer adhered to an outer surface of the second outer layer of the multilayer film. In one embodiment, the first metal layer, the second metal layer or both metal layers can be copper. In one embodiment, a metal-clad laminate of the present invention comprising a double-side copper-clad can be prepared by laminating copper foil to both sides of the single-layer or multilayer film.

### Applications

In one embodiment, a polymer film with high tensile strength and low CTE can be used in electronic device applications, such as flexible device layers for electronic device or a coverlay for a printed circuit board or other electronic components in an electronic device, providing protection from physical damage, oxidation and other contaminants that may adversely affect the function of the electronic components. In one embodiment polymer films can be used for flexible display applications, such as for thin-film transistor (TFT) substrates in organic light-emitting diode (OLED) displays, electronic paper (E-paper) and touch sensor panels (TSPs) for displays.

In one embodiment, a polymer film with low color and high tensile strength can be used for a number of layers in electronic device applications, such as in an organic electronic device, where a combination of good optical and mechanical properties is desirable. Nonlimiting examples of such layers include device substrates, touch panels, substrates for color filter sheets, cover films, and others. The particular materials' properties requirements for each application are unique and may be addressed by appropriate composition(s) and processing condition(s) for the polymer films disclosed herein. Organic electronic devices that may benefit from having a coated film include, but are not limited to, (1) devices that convert electrical energy into radiation (e.g., a light-emitting diode, light emitting diode display, lighting device, luminaire, or diode laser), (2) devices that detect signals through electronics processes (e.g., photodetectors, photoconductive cells, photoresistors, photoswitches, phototransistors, phototubes, IR detectors, biosensors), (3) devices that convert radiation into electrical energy, (e.g., a photovoltaic device or solar cell), (4) devices that convert light of one wavelength to light of a longer wavelength, (e.g., a down-converting phosphor device); and (5) devices that include one or more electronic components that include one or more organic semi-conductor layers (e.g., a transistor or diode).

In one embodiment, a metal-clad laminate having a polymer film is particularly useful for die pad bonding of flexible print connection boards or semiconductor devices or packaging materials for CSP (chip scale package), COF (chip on film), COL (chip on lead), LOC (lead on chip), MCM (multi-chip module), BGA (ball grid array or micro-ball grid array), and/or TAB (tape automated bonding).

In another embodiment, the polymer films are useful for wafer level integrated circuit packaging, where a composite is made using a polymer film interposed between a conductive layer (typically a metal) having a thickness of less than 100 μm, and a wafer comprising a plurality of integrated circuit dies. In one (wafer level integrated circuit packaging) embodiment, the conductive passageway is connected to the dies by a conductive passageway, such as a wire bond, a conductive metal, a solder bump or the like.

The advantageous properties of this invention can be observed by reference to the following examples that illustrate, but do not limit, the invention. All parts and percentages are by weight unless otherwise indicated.

### EXAMPLES

### Test Methods

### Measurement of CIE L*, a*, b* Color

Color measurements were performed using a ColorQuest® XE dual-beam spectrophotometer (Hunter Associates Laboratory, Inc., Reston, VA), using D65 illumination and 10-degree observer, in total transmission mode over a wavelength range of 380 to 780 nm. Percent haze and transmittance were also measured using this instrument.

### Yellowness Index

Yellowness Index (YI) was measured using the procedure described by ASTM E313.

### Glass Transition Temperature

Glass transition temperature (Tg) was measured using dynamic mechanical analysis (Q800 DMA, TA Instrument) and is determined by the tan delta peak.

### Coefficient of Thermal Expansion

Coefficient of thermal expansion (CTE), or coefficient of linear expansion, was measured using dynamic mechanical analysis (Q800 DMA, TA Instrument). The CTE was measured over a temperature range of 50 to 250° C. in both the machine direction (MD) and the transverse direction (TD). In both cases, the sample was cycled through the temperature range twice, with the CTE of the second cycle reported for the measurement.

### Tensile Modulus

Tensile modulus was measured using the ASTM D882 test method.

### Thickness

Film thickness was determined by measuring 5 positions across the profile of the film using a contact-type FISCHERSCOPE MMS PC2 modular measurement system thickness gauge (Fisher Technology Inc., Windsor, CT).

### Example 1

A cyclohexane dicarbonyl chloride (CHDC) monomer was prepared. In a dry box, in a 1 L round-bottomed flask fitted with a 20″ Vigreaux column, to 24.00 g of trans-1,4-cyclohexanedicarboxylic acid in 200 ml of dichloromethane (DCM) was added 300 ml of 2 M oxalyl chloride in DCM, followed by 2.0 ml of dimethyl formamide (DMF). The mixture was heated at 55.5° C. After 23 hr, the reaction was cooled slowly to room temperature. The orange reaction was removed from the glove box and concentrated in vacuo to give a dark burnt orange solid. The crude product was recrystallized from 310 ml hexanes (in the dry box). Copper-colored, undissolved solids were filtered off prior to crystallization. The product was filtered and washed with ˜100 ml of hexanes to give the product as very light tan needles which were dried in the antechamber overnight. Yield: 15.2 g (51.5%) of the product as very light tan needles.

For Example 1 (E1), a poly(amide-amic) acid was prepared having a monomer composition of CHDC 0.5/6FDA 0.5//TFMB 1.0. A 4-necked, 1 L reaction vessel with a PTFE stirring shaft, o-ring, and bearing assembly was heated under a sweep of nitrogen under vacuum in an oven at 159° C. overnight. The reactor assembly was removed from the oven, assembled while hot, and nitrogen was passed through the assembly and allowed to cool to room temperature while passing a stream of nitrogen through the assembly. A solution of 34.925 g of 2,2′-bis(trifluoromethyl)benzidine (TFMB, Seika Corp., Wakayama Seika Kogyo Co., LTD., Japan) in 454 g anhydrous dimethyl acetate (DMAc) was added into the reaction assembly and heated to 40° C. After the thermometer reached an external temperature of 42.2° C., a solution of 11.288 g of CHDC (as prepared above) and 23.985 g of 4,4′-(hexafluoroisopropylidene)diphthalic anhydride (6FDA Daikin America, Orangeburg, NY) in 114 g of anhydrous DMAc was added via addition funnel, dropwise. After heating for 1 hr, the reaction was stirred at room temperature. After stirring overnight, an additional 0.192 g of 6FDA in 2.66 g of DMAc was added. After stirring overnight, an additional 0.096 g of 6FDA in 0.96 g of DMAc was added. After stirring overnight, an additional 0.192 g of 6FDA in 1.6 g of DMAc was added. After 4 days, an additional 706 mg of 6FDA was added. The reaction was 101.4% dicarbonyl-containing monomers. After 2 days, the viscosity was 3,437 centipoise.

The solution was cast onto a glass substrate at 25° C. to produce ˜2 mil films. A doctor blade was used with a 40 mm clearance to produce ˜2 mil films after curing. The film on the glass substrate was heated to 50° C. for 30 min then to 90° C. for 30 min on a hotplate, and it was subsequently cooled to room temperature. The film was released using a razor and mounted onto a 4×8 inch pin frame and placed in a furnace (Carbolite Gero, Sheffield, UK). The furnace was then purged with nitrogen and heated according to the following temperature protocol:
25 to 90° C. (7° C./min), hold at 90° C. for 5 minutes;
90 to 150° C. (7° C./min), hold at 150° C. for 10 minutes;
150 to 320° C. (7° C./min), hold at 320° C. for 5 minutes;
   The film was removed from the furnace after heating to 320° C. for 5 minutes and allowed to cool in air.

### Example 2

For Example 2 (E2), a substantially imidized solution was formed by the following procedure. 200 g of the poly(amide-amic) acid solution described in E1 was combined with 8.87 g of beta-picoline (Aldrich Chemicals, St. Louis, MO) and 9.72 g of acetic anhydride (Aldrich). The solution was heated to 80° C. for 2 hr before allowing to cool to room temperature.

The solution was precipitated and washed with methanol by combining 200 g of the solution described above with 500 ml of methanol. The material was ground in a blender, filtered and washed twice with an equal volume of methanol. The final powder was dried at 50° C. under vacuum for 24 hr.

The polymer resin was combined with DMAc to create a 16.2 wt % solution for casting. The solution was cast onto a glass substrate and heated on a hotplate as described above for E1. The film was released using a razor and mounted onto a 4×8 inch pin frame and placed in the furnace. The furnace was then purged with nitrogen and heated according to the following temperature protocol:
25 to 90° C. (7° C./min), hold at 90° C. for 5 minutes;
90 to 150° C. (7° C./min), hold at 150° C. for 10 minutes;
150 to 250° C. (7° C./min), hold at 250° C. for 20 minutes;
   The film was removed from the furnace after heating to 250° C. for 20 minutes and allowed to cool in air.

### Example 3

For Example 3 (E3), the polymer resin as described above for E2 was used. An ˜30 wt % nanocolloid of silicon oxide was created in DMAc by reacting with trimethoxyphenylsilane. 2.5 g of the colloid was combined with a solution consisting of 2.25 g of the dried polymer and 16.4545 g of DMAc.

The solution was cast onto a glass substrate and heated on a hotplate as described above for E1. The film was released using a razor and mounted onto a 4×8 inch pin frame and placed in the furnace. The furnace was then purged with nitrogen and heated to a final temperature of 250° C. as described above for E2. The film was removed from the furnace after 20 minutes at 250° C. and allowed to cool in air.

### Example 4

A CHDC monomer was prepared. In a dry box, in a 1 L round-bottomed flask fitted with a 20″ Vigreaux column, to 31.72 g of trans-1,4-cyclohexanedicarboxylic acid in 500 ml of DCM, was added 100 g of oxalyl chloride, then 1.4 ml of DMF. The mixture was heated at 40° C. for 2 h 35 min then at 45° C. for 2 hr and allowed to cool to room temperature overnight. The light-yellow reaction was concentrated in vacuo to give an off-white solid which was recrystallized in a glove box (some undissolved solids were present) from ˜700 ml hexanes. Burnt orange-colored undissolved solids were filtered off while still hot and the filtrate allowed to cool. The product was filtered, washing with 100 ml hexanes to give off-white crystals. The product was dried at room temperature in a glove box antechamber overnight then at 50° C. for 17.5 hr under house vacuum. Yield: 15.77 g (41%) of the product as a light pink/tan off-white solid.

For Example 4 (E4), a poly(amide-amic) acid was prepared having a monomer composition of CHDC 0.7/6FDA 0.3//TFMB 1.0. All glassware was dried at 160° C. overnight. In a nitrogen purged dry box and a 250 ml reaction vessel, 5 g of TFMB was combined with 60.3 g of DMAc. In four aliquots over the course of 1 hr were added 2.258 g of the CHDC and 2.056 g of 6FDA along with an additional 15.1 g of DMAc. An additional 0.028 g of 6FDA dissolved in 0.5 g of DMAc was added. After 24 hr, the viscosity of the solution was 92 poise.

84.67 g of the poly(amide-amic) acid solution was used. An additional 20 g of DMAc was added to the solution which was heated to 40° C. along with 3.99 g of acetic anhydride and 3.64 g of beta-picoline. The solution was then heated to 80° C. for 2 hr. After allowing the solution to cool to room temperature, it was precipitated in methanol, washed and dried to produce the polyimide resin.

The solution was precipitated and washed with methanol by combining 200 g of the solution described above with 500 ml of methanol. The material was ground in a blender, filtered and washed twice with an equal volume of methanol. The final powder was dried at 50° C. under vacuum for 24 hr.

A 14 wt % solution of the polymer resin in DMAc was used to cast films. The solution was cast onto a glass substrate at 25° C. to produce ˜2 mil films. A doctor blade was used with a 25 mm clearance to produce ˜2 mil films after curing. The film on the glass substrate was heated to 50° C. for 30 min then to 90° C. for 30 min on a hotplate, and it was subsequently cooled to room temperature. The film was released using a razor and mounted onto a 4×8 inch pin frame and placed in a furnace. The furnace was then purged with nitrogen and heated to a final temperature of 250° C. as described above for E2. The film was removed from the furnace after 20 minutes at 250° C. and allowed to cool in air.

### Example 5

The CHDC monomer as described above in E4 was used.

For Example 5 (E5), a poly(amide-amic) acid was prepared having a monomer composition of CHDC 0.8/6FDA 0.2//TFMB 1.0. All glassware was dried at 160° C. overnight. In a nitrogen purged dry box and a 250 ml reaction vessel, 3.0 g of TFMB was combined with 34.8 g of DMAc. In four aliquots over the course of 1 hr were added 1.548 g of the CHDC and 0.822 g of 6FDA along with an additional 8.7 g of DMAc. An additional 0.028 g of 6FDA dissolved in 0.5 g of DMAc was added. After 24 hr, the viscosity of the solution was 310 poise.

40 g of DMAc was added to the polymer solution to create a 10 wt % solids solution.

The solution was cast onto a PET substrate at 25° C. to produce ˜2 mil films. A doctor blade was used with a 20 mm clearance to produce ˜2 mil films after curing. The film on the PET substrate was heated to 50° C. for 30 min then to 90° C. for 30 min on a hotplate, and it was subsequently cooled to room temperature. The film was released using a razor and mounted onto a 4×8 inch pin frame and placed in a furnace. The furnace was then purged with nitrogen and heated according to the following temperature protocol:
25 to 90° C. (7° C./min), hold at 90° C. for 5 minutes;
90 to 150° C. (7° C./min), hold at 150° C. for 10 minutes;
150 to 300° C. (7° C./min), hold at 300° C. for 5 minutes;
   The film was removed from the furnace after heating to 300° C. for 5 minutes and allowed to cool in air.

### Example 6

For Example 6 (E6), the same poly(amide-amic) acid solution as described above for E5 was used.

48.82 g of the poly(amide-amic) acid solution was used. An additional 56.0 g of DMAc was added to the solution which was heated to 40° C. along with 3.83 g of acetic anhydride and 3.49 g of beta-picoline. The solution was then heated to 80° C. for 2 hr. After allowing the solution to cool to room temperature, it was precipitated in methanol, washed and dried to produce the polyimide resin.

The solution was precipitated with a combination of water and washed with methanol by adding 100 g of polymer solution to a blender. 200 g of methanol was used as an antisolvent. The precipitate was washed using additional methanol and allowed to dry on a Buchner funnel.

A 11 wt % solution of the resin in DMAc was used to cast films.

The solution was cast onto a glass substrate at 25° C. to produce ˜2 mil films. The coating solution of composition was cast on a glass substrate at 25° C. using a doctor blade with a 40 mm clearance to produce ˜2 mil film after curing. The film on the glass substrate was heated to 65° C. for 20 min then to 85° C. for 30 min on a hotplate. The film was allowed to cool to room temperature. The film was released using a razor and mounted onto a 4×8 inch pin frame and placed in a furnace. The furnace was then purged with nitrogen and heated to a final temperature of 250° C. as described above for E2. The film was removed from the furnace after 20 minutes at 250° C. and allowed to cool in air.

### Example 7

The CHDC monomer as described above in E4 was used.

For Example 7 (E7), a poly(amide-amic) acid was prepared having a monomer composition of CHDC 0.5/BPDA 0.5//TFMB 1.0. All glassware was dried at 160° C. overnight. In a nitrogen purged dry box and a 250 ml reaction vessel, 5.0 g of TFMB was combined with 57.5 g of DMAc. In four aliquots over the course of 1 hr were added 1.613 g of the CHDC and 2.269 g of 3,3,4,4-biphenyltetracarboxylic anhydride (BPDA, Mitsubishi Chemicals America, Inc., Charlotte, NC) along with an additional 14.4 g of DMAc. An additional 0.072 g of 6FDA dissolved in 0.5 g of DMAc was added. After 24 hr, the viscosity of the solution was 75 poise.

80.75 g of the poly(amide-amic) acid solution was used. 6.42 g of acetic anhydride and 5.86 g of beta-picoline were added, and the solution was then heated to 80° C. for 2 hr. After allowing the solution to cool to room temperature, it was precipitated in methanol, washed and dried to produce the polyimide resin.

The solution was precipitated and washed with methanol by combining 200 g of the solution described above with 500 ml of methanol. The material was ground in a blender, filtered and washed twice with an equal volume of methanol. The final powder was dried at 50° C. under vacuum for 24 hr.

A 5 wt % solution of the resin in DMAc was prepared. The solution was cast onto a glass substrate at 25° C. to produce ˜2 mil films. The coating solution of composition was cast on a glass substrate at 25° C. using a doctor blade with a 50 mm clearance to produce ˜2 mil film after curing. The film on the glass substrate was heated to 50° C. for 30 min then to 90° C. for 30 min on a hotplate. The film was allowed to cool to room temperature. The film was released using a razor and mounted onto a 4×8 inch pin frame and placed in a furnace. The furnace was then purged with nitrogen and heated to a final temperature of 250° C. as described above for E2. The film was removed from the furnace after 20 minutes at 250° C. and allowed to cool in air.

### Example 8

A CHDC monomer was prepared. In a dry box, in a 1 L round-bottomed flask fitted with a 20″ Vigreaux column, to 23.9 g of trans-1,4-cyclohexanedicarboxylic acid in 200 ml of DCM, was added 300 g of 2 M oxalyl chloride in DCM, then 20 drops of DMF. The mixture was heated at 57° C. for 3 hr then at 54° C. for 3.5 hr and allowed to cool to room temperature overnight. The gold-colored solution was removed from the dry box and concentrated in vacuo to give a tan solid. After the solid appeared dry, it remained under 40 mbar at 62° C. for 15 min. 170 ml was retrieved in the rotovap collection flask. The crude product was recrystallized from 250 ml hexanes in the dry box. The contents were filtered while hot (copper-colored undissolved solids remained in the reaction flask) and the product crashed out in the filtrate flask as it was filtered. The product (off-white needles) was filtered and washed with ˜100 ml of hexanes and then dried in a dry box antechamber overnight. After ˜67 hr 40 min, the product was removed from antechamber. Yield:15.72 g (54%) of the product as off-white needles.

For Example 8 (E8), a poly(amide-amic) acid was prepared having a monomer composition of CHDC 0.5/TPC 0.3/6FDA 0.2//TFMB 1.0. All glassware was dried at 160° C. overnight. In a nitrogen purged dry box and a 250 ml reaction vessel, 4.0 g of TFMB was combined with 46.2 g of DMAc. In four aliquots over the course of 1 hr were added 1.290 g of the CHDC, 1.096 g 6FDA and 0.752 g terephthaloyl chloride (Aldrich Chemicals, St. Louis, MO) along with an additional 11.6 g of DMAc. 40 g of DMAc was added to the solution.

The polymer solution described above was used for film formation.

The solution was cast onto a glass substrate and heated on a hotplate as described above for E7. The film was released using a razor and mounted onto a 4×8 inch pin frame and placed in a furnace. The furnace was then purged with nitrogen and heated to a final temperature of 300° C. as described above for E5. The film was removed from the furnace after 5 minutes at 300° C. and allowed to cool in air.

### Example 9

The CHDC monomer as described above in E8 was used.

For Example 9 (E9), a poly(amide-amic) acid was prepared having a monomer composition of CHDC 0.5/CBDA 0.3/6FDA 0.2//TFMB 1.0. All glassware was dried at 160° C. overnight. In a nitrogen purged dry box and a 500 ml reaction vessel, 30.0 g of TFMB was combined with 345.3 g of DMAc. In four aliquots over the course of 1 hr were added 9.676 g of the CHDC, 8.224 g 6FDA and 5.442 g of cyclobutane-1,2,3,4-tetracarboxylic dianhydride (CBDA, Wilshire Technologies, Princeton NJ) along with an additional 86.3 g of DMAc. An additional 0.575 g of 6FDA was added during five separate additions along with about 11 g of DMAc. The final viscosity was approximately 15 poise.

484.92 g of the poly(amide-amic) acid solution was used. 23.78 g of acetic anhydride and 21.69 g of beta-picoline were added. The solution was then heated to 80° C. for 2 hr. After allowing the solution to cool to room temperature, the poly(amide-imide) solution was used for film formation.

The solution was cast onto a PET at 25° C. to produce ˜2 mil films. The coating solution of composition was cast on a glass substrate at 25° C. using a doctor blade with a 10 mm clearance to produce ˜2 mil film after curing. The film on the PET substrate was heated to 50° C. for 30 min then to 90° C. for 30 min on a hotplate. The film was allowed to cool to room temperature. The film was released using a razor and mounted onto a 4×8 inch pin frame and placed in a furnace. The furnace was then purged with nitrogen and heated according to the following temperature protocol:
25 to 90° C. (7° C./min), hold at 90° C. for 5 minutes;
90 to 150° C. (7° C./min), hold at 150° C. for 10 minutes;
150 to 230° C. (7° C./min), hold at 230° C. for 15 minutes;
   The film was removed from the furnace after heating to 230° C. for 15 minutes and allowed to cool in air.

Table 1 summarizes the optical data for the films made in E1-E9, and Table 2 summarizes the thermal and mechanical properties of the films.

TABLE 1

| | | T | Haze | | | | | t |
|---|---|---|---|---|---|---|---|---|
| Example | Composition | (%) | (%) | L* | a* | b* | YI | (μm) |
| E1 | CHDC 0.5/6FDA 0.5//TFMB 1.0 | 91.58 | 0.52 | 96.00 | −0.30 | 2.12 | 3.78 | 61.7 |
| E2 | CHDC 0.5/6FDA 0.5//TFMB 1.0 | 90.80 | 0.59 | 96.20 | −0.04 | 0.71 | 1.32 | 35.6 |
| E3 | CHDC 0.5/6FDA 0.5//TFMB 1.0 | 92.44 | 2.15 | 96.08 | −0.26 | 2.04 | 3.67 | 43.2 |
| | (with SiO2 nanocolloid) | | | | | | | |
| E4 | CHDC 0.7/6FDA 0.3//TFMB 1.0 | 92.47 | 1.39 | 96.55 | −0.10 | 0.91 | 1.65 | 45.2 |
| E5 | CHDC 0.8/6FDA 0.2//TFMB 1.0 | 86.80 | 1.22 | 94.20 | −0.17 | 4.08 | 7.65 | 13.2 |
| E6 | CHDC 0.8/6FDA 0.2//TFMB 1.0 | 92.00 | 0.89 | 95.89 | −0.15 | 1.35 | 2.45 | 23.1 |
| E7 | CHDC 0.5/BPDA 0.5//TFMB 1.0 | 90.10 | 3.45 | 94.89 | −0.64 | 2.87 | 4.98 | 17.3 |
| E8 | CHDC0.5/TPC 0.3/6FDA 0.2//TFMB 1.0 | 85.10 | 7.13 | 94.18 | −0.67 | 7.05 | 12.71 | |
| E9 | CHDC 0.5/CBDA 0.3/6FDA 0.2//TFMB 1.0 | 91.86 | 1.09 | 95.50 | −0.15 | 2.24 | 4.14 | 45.5 |

TABLE 2

| | | T_{g} | CTE | CTE | Tensile Modulus |
|---|---|---|---|---|---|
| Example | Composition | (° C.) | (MD) | (TD) | (MD, GPa) |
| E1 | CHDC 0.5/6FDA 0.5//TFMB 1.0 | 353 | 65.62 | 67.89 | |
| E2 | CHDC 0.5/6FDA 0.5//TFMB 1.0 | 362 | 45.34 | 46.90 | 4.27 |
| E3 | CHDC 0.5/6FDA 0.5//TFMB 1.0 | 363 | 50.68 | 49.63 | 4.86 |
| | (with SiO2 nanocolloid) | | | | |
| E4 | CHDC 0.7/6FDA 0.3//TFMB 1.0 | 366 | 29.62 | 30.13 | 3.46 |
| E5 | CHDC 0.8/6FDA 0.2//TFMB 1.0 | 355 | 23.34 | 20.52 | 5.06 |
| E6 | CHDC 0.8/6FDA 0.2//TFMB 1.0 | 359 | 20.39 | 18.29 | 5.34 |
| E7 | CHDC 0.5/BPDA 0.5//TFMB 1.0 | 335 | 7.52 | 8.26 | 6.67 |
| E8 | CHDC0.5/TPC 0.3/6FDA 0.2//TFMB 1.0 | 351 | 18.48 | 42.26 | 5.76 |
| E9 | CHDC 0.5/CBDA 0.3/6FDA 0.2//TFMB 1.0 | 385 | 34.11 | 29.28 | 5.42 |

What is claimed is:

1. A dicarbonyl halide having formula I: 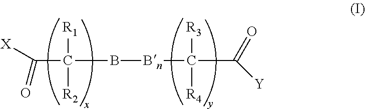
wherein:
X and Y are the same or different at each occurrence and are independently selected from the group consisting of fluorine, chlorine and bromine;
R₁-R₄ are the same or different at each occurrence and are independently selected from the group consisting of hydrogen and alkyl, wherein alkyl is C1 to C12 and linear or branched;
B and B′ are the same or different at each occurrence and are an alicyclic group or an aromatic group with the proviso that at least one of B and B′ is an alicyclic group;
x and y are the same or different and are an integer from 0 to 12; and
n is an integer from 0 to 4.

2. The dicarbonyl halide of claim 1, wherein the alicyclic group of each B and B′ is selected from the group consisting of a phenyl group, a saturated C3 to C16 alicyclic group, an unsaturated, non-aromatic C5 to C22 alicyclic group and a C7 to C8 bicyclic group.

3. The dicarbonyl halide of claim 1, wherein X=Y=chlorine.

4. The dicarbonyl halide of claim 3, wherein n=0 and B is a saturated C4 to C9 alicyclic group.

5. The dicarbonyl halide of claim 4, wherein B is a saturated C6 alicyclic group.

6. A polymer composition derived from the dicarbonyl halide of claim 1, a dianhydride and a diamine, wherein the polymer composition is a poly(amide-imide) or a poly(amide-ester-imide), wherein the poly(amide-ester-imide) is further derived from a polyol.

7. The polymer composition of claim 6, wherein the dianhydride is selected form the group consisting of 3,3′,4,4′-biphenyltetracarboxylic dianhydride, cyclobutane-1,2,3,4-tetracarboxylic dianhydride, 3,3′,4,4′-benzophenonetetracarboxylic dianhydride, 4,4′-(hexafluoroisopropylidene)diphthalic anhydride, 9,9-bis(trifluoromethyl)-2,3,6,7-xanthenetetracarboxylic dianhydride and mixtures thereof.

8. The polymer composition of claim 6, wherein the diamine is selected from the group consisting of 2,2′-bis(trifluoromethyl)benzidine, p-phenylenediamine, 4,4′-bis-(aminophenoxy)biphenyl, 1,3-bis(4-aminophenoxy) benzene, trans-1,4-cyclohexanediamine, 1,6-diaminohexane, 1,4-diaminobutane, 1,5 diaminopentane, 1,7-diaminoheptane and mixtures thereof.

9. A polymer film comprising the polymer composition of claim 6.

10. The polymer film of claim 9, wherein the polymer film has a tensile modulus of 4 GPa or more.

11. The polymer film of claim 9, wherein the polymer film has a coefficient of thermal expansion of 50 ppm/° C. or less over a temperature range of 50 to 250° C.

12. The polymer film of claim 9, wherein the polymer film has a b* of 1.25 or less and a yellowness index of 2.25 or less for a film having a thickness of 25 μm.

13. An electronic device comprising the polymer film of claim 9.

14. The electronic device of claim 13, wherein the electronic device is a flexible display.

15. A metal-clad laminate comprising the polymer film of claim 9.
